(19) United States
(12) Patent Application Publication
(10) Pub. No.: US 2008/0216564 A1
(43) Pub. Date: Sep. 11, 2008

(54) Biosensors for Detecting Bond Rupture

(76) Inventors: Yong Yuan, Lower Hutt (NZ); Michael Andrews, Pautahanui (NZ); W Michael Arnold, Wellington (NZ); Barry Marlow, Lower Hutt (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/592,562

(22) PCT Filed: Mar. 21, 2005
(86) PCT No.: PCT/NZ2005/000051

§ 371 Date: May 7, 2008

(30) Foreign Application Priority Data
Mar. 19, 2004 (NZ) 528338

(51) Int. Cl. G01N 29/00; G01N 21/01; C12Q 1/00

(52) U.S. Cl. 73/61.75; 422/82.05; 435/4

(57) ABSTRACT

A biosensor comprises a surface onto which bio-macromolecules are bound, the surface and bio-macromolecules being immersed in liquid, a bond rupture detector associated with the surface and arranged to detect the rupture of bonds between the bio-macromolecules and a target substance (6), and an oscillator (8) associated with the liquid and spaced from the surface and arranged to produce oscillations in the liquid to cause bonds between the bio-macromolecules and the target substance (6) to rupture.

### FIELD OF INVENTION

The invention relates to biosensors for detecting a variety of chemical and biological agents and in particular to biosensors for detecting a variety of chemical and biological agents with a separate detector and oscillator.

### BACKGROUND

In current New Zealand medical practices when a patient is ill a swab is taken and analysed in a laboratory. For example to identify a strain of influenza in a patient an aliquot of appropriate fluid is incubated with the purified antibody to a characteristic molecule or marker on the surface of the influenza virus. By means of amplifying agents and secondary antibodies, which are tagged with enzymes or radioisotopes, the concentration of the virus can be determined. Such assays, while quite sensitive, take a minimum of several hours but often considerably longer. Thus it may be several hours or even a day before an accurate diagnosis is available for the patient. Even greater delays may be involved if transport of a sample to a city laboratory from a remote area is required.

Medical diagnostic tests use recognition and binding between two biological molecules. However, detection methods using immuno-reactions such as antibody-antigen binding can have greater application, as long as a specific binding partner can be generated to recognize the target molecule (such as a protein).

Medical technology conventionally attaches (“immobilises”) one of the molecules to a surface, which is then exposed to a liquid containing the analyte, which if present, binds selectively to the surface via its binding partner.

There are several methods of detecting the occurrence of binding, and the number of bound sites. The analyte may be used to competitively displace pre-bound, but labelled particles, for example those tagged fluorescently, and detection is signalled by a change in fluorescence. A common assay is the enzyme based ELISA method, which though sensitive, is generally slower than the basic binding process, which occurs within minutes. In both these methods, specially modified marker molecules are required for detection.

One method capable of detecting changes in surface binding without requiring labelled chemicals uses the optical phenomenon of surface plasmon resonance. This probes a liquid interface above, for example a glass slide, using evanescent waves, and has the sensitivity to detect changes in biological surface binding with a fast reaction time.

All affinity binding methods suffer from non-specific adsorption, the process by which proteins other than those sought attach themselves to a surface, on sites other than the prepared “capture” molecules. This is a limiting factor with regard to detection threshold.

One approach to circumventing non-specific binding involves a bio-macromolecule bound to a quartz crystal microbalance. The bio-macromolecule is brought into contact with a target substance to which the bio-macromolecule binds. The quartz crystal microbalance is then oscillated with increasing amplitude until the bonds between the target substance and the bio-macromolecule break. During oscillation non-specifically bound material will be removed before the target substance. The bond rupture is detected and hence the presence of the target substance is confirmed. If detection of target substances depends on the high Q of the quartz resonator, it is expected that the sensitivity will be decreased by operation in liquids due to the high viscoelastic damping known to be produced. A liquid compatible system is important since that is the natural environment of the bio-macromolecules.

### SUMMARY OF INVENTION

It is the object of the present invention to provide a biosensor with a separate oscillator and rupture detector, or to at least provide the public with a useful choice.

In broad terms in one aspect the invention comprises a biosensor including a surface onto which bio-macromolecules are bound, the surface and bio-macromolecules being immersed in liquid, a bond rupture detector associated with the surface and arranged to detect the rupture of bonds between the bio-macromolecules and a target substance, and an oscillator associated with the liquid and spaced from the surface and arranged to produce waves in the liquid to cause bonds between the bio-macromolecules and the target substance to rupture.

In one embodiment the detector is a surface plasmon resonance detector that detects, when bonds between the bio-macromolecules and the target substance rupture, a change in the angle of reflected light that has minimum reflectance. In another embodiment the detector detects acoustic emissions produced when bonds between the bio-macromolecules and target substance rupture. In another embodiment any other suitable detector may be used.

The oscillator may be any device suitable for providing oscillating motion in the liquid (“waves”). The oscillator may be an acoustic oscillator, a piezoelectric device, a mechanical resonator or a microcantilever. The liquid may also be moved electrophoretically or by magnetohydrodynamics. Preferably the waves are ultrasonic in frequency.

The oscillator may be arranged to provide waves at a predetermined frequency, or may be arranged to provide waves over a range of frequencies. The amplitude of the waves may be constant or may change, for example the amplitude of the waves may increase at a constant rate.

Preferably a self assembled monolayer is provided to bind the bio-macromolecules to the surface.

Preferably the surface is coated in gold or silver. Alternatively any suitable metals may be used as the surface or as a coating on the surface.

In one embodiment more than one surface is provided with bio-macromolecules that bind to different target substances provided on each surface.

In broad terms in another aspect the invention comprises a method of detecting a target substance including the steps of providing a biosensor including at least one surface onto which bio-macromolecules are bound, a bond rupture detector associated with the surface and arranged to detect the rupture of bonds between the bio-macromolecules and a target substance, and an oscillator separate from the surface and arranged to produce waves to cause bonds between the bio-macromolecules and the target substance to rupture, bringing the biosensor into contact with a test fluid that may contain the target substance, using the oscillator to provide waves directed at the surface and using the detector to detect whether any bonds rupture and comparing the parameter of the oscillator when the bonds rupture with stored data and where during operations of the biosensor the surface of the biosensor and the oscillator are immersed in liquid.

### BRIEF DESCRIPTION OF DRAWINGS

The invention will be further described by way of example only and without intending to be limiting with reference to the following drawings, wherein:

FIG. 1A shows the sensor surface of a biosensor of the invention;

FIG. 1B shows the sensor surface of a biosensor of the invention after contact with a test fluid;

FIG. 1C shows one embodiment of biosensor of the invention during oscillation when non-specific binding molecules are separated from the bio-macromolecules;

FIG. 1D shows one embodiment of biosensor of the invention during oscillation to rupture bonds between bio-macromolecules on the sensor surface and molecules bonded to the bio-macromolecules;

FIG. 2 shows the binding of bio-macromolecules to the biosensor;

FIG. 3 is a block diagram of the biosensor of the invention.

FIG. 4 is a schematic of one embodiment of biosensor of the invention;

FIG. 5 shows the molecular structure of Biotin-PEO3-Amine;

FIG. 6 shows the change in refractive index of an SPR detector as a self-assembled monolayer is formed on a substrate and buffered; and

FIG. 7 shows the change in refractive index of an SPR detector as a target species binds to the self-assembled monolayer with subsequent rupture of the bonds between the target species and the self-assembled monolayer.

### DETAILED DESCRIPTION

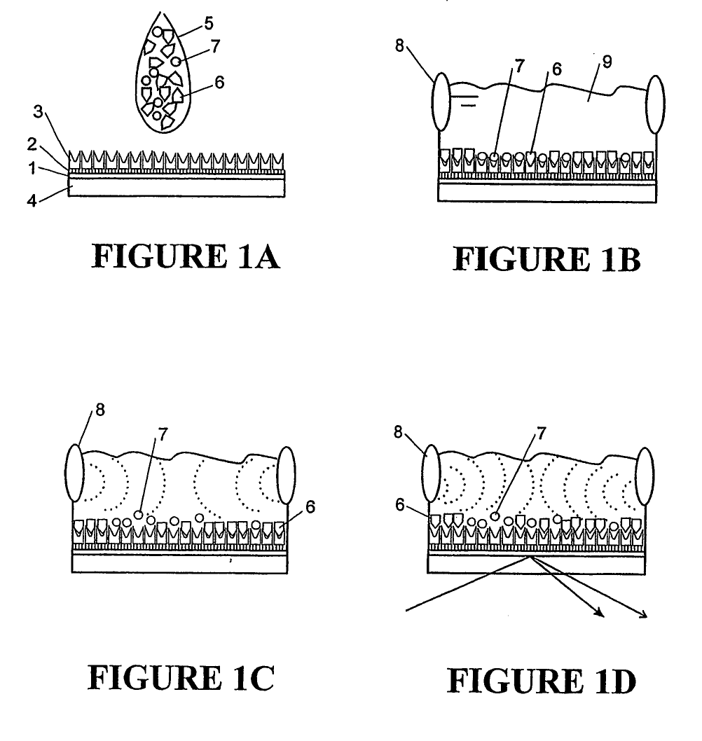

FIG. 1A shows a cross section of a sensor surface of a preferred form of the biosensor of the invention. The sensor surface includes a substrate 4, sensor surface metal layer 1, self assembled monolayer 2 and bio-macromolecule layer 3.

It will be appreciated that the target substance and bio-macromolecule may be inverted. For example, the bio-macromolecule may be an antibody where the target substance is an antigen and vice versa. Bio-macromolecule layer 3 is chosen as a substance that will form a bond with the target substance. For example if the target substance is an antigen, bio-macromolecule layer 3 can be a layer of antibodies. Other examples include using a toxin (for example tetrodotoxin, saxitoxin or brevetoxin) as bio-macromolecule layer 3 to test for sodium ion channel membrane fragments, using an inhibitor (for example okadaic acid, proteins or metals) as bio-macromolecule layer 3 to test for enzymes, using lectins as bio-macromolecule layer 3 to test for specific carbohydrates on a cell surface, or using a virus as bio-macromolecule layer 3 to test for receptor substances on a cell surface. Alternatively the bio-macromolecule layer 3 may comprise a layer of nucleic acid aptamers to test for specific ligands. A further alternative embodiment involves using oligonucleotides as bio-macromolecule layer 3 to test for complementary RNA or DNA molecules. In a more specific embodiment bio-macromolecule layer 3 may be antibody AA5H or B017 that are used to detect the presence of influenza A and B viruses respectively.

A self assembled monolayer 2 forms the layer between the sensor surface 1 and bio-macromolecule layer 3. The self assembled monolayer bonds to both the sensor surface metal layer 1 and the bio-macromolecule layer 3. The composition of the self assembled monolayer may depend on the bio-macromolecule layer 3 that will be bound to the self assembled monolayer.

The sensor surface metal layer 1 may be any metal layer onto which a self assembled monolayer may be formed. Typically the metal layer is formed from gold.

Substrate 4 may be formed from any suitable material. In the preferred embodiment surface plasmon resonance is used to detect bond rupture. In this embodiment the substrate may be formed from glass or plastics. In this embodiment the substrate must be transparent.

Sample 5 is shown above the biosensor in FIG. 1A. Sample 5 contains molecules of a target substance 6 as well as other molecules including molecules 7. Molecules 7 will also bind to bio-macromolecules 3 but, in contrast to molecules 6, do not form strong bonds with the bio-macromolecules.

FIG. 1B shows the sensor surface of the biosensor after contact with a test fluid. The test fluid may or may not contain the target substance but in the example shown does contain the target substance. The bio-macromolecule bonded to the sensor surface is chosen for its binding selectivity for the target substance. When the test fluid contacts the sensor surface any molecules of the target substance present in the test fluid will bind with the bio-macromolecule 3 of the sensor surface. Other substances or species present in the test fluid may also bind with the bio-macromolecule but (if the best bio-macromolecule is chosen) will form weaker bonds with the bio-macromolecule than will the target substance.

As can be seen in FIG. 1B when the sample is provided on the sensor the sensor surface is immersed in liquid 9. Liquid 9 may be water or any other liquid suitable for use in the sensor. Liquid 9 may flow across the sensor surface. In other embodiment the liquid may not flow across the sensor surface. The oscillator 8 is also at least partially immersed in liquid. Oscillator 8 may be an acoustic oscillator, a piezoelectric device, a mechanical resonator or a microcantilever. Oscillator 8 may oscillate the liquid or move the liquid electrophoretically or by magnetohydrodynamics.

After the test fluid has been in contact with the sensor surface, waves are produced from oscillator 8 as shown in FIG. 1C. Both the amplitude and frequency of waves from the oscillator may be varied. As the waves are applied to the sensor surface substances that are not bonded to the sensor surface are shaken off the sensor surface as shown in FIG. 1C. Eventually the bonds between the bio-macromolecules and substances from the test fluid bonded to the bio-macromolecules will rupture as shown in FIG. 1D. FIG. 1D also shows a change in resonance angle that will be detected by surface plasmon resonance as the bonds between the target substance and the biosensor molecules rupture.

The relative motion between the “target” particles and the surface provides the bond breaking energy. The particles are caused to oscillate by driving them via liquid movement, rather than oscillating the surface. In the preferred embodiment bond rupture is detected by step changes in the surface plasmon resonance (SPR) angle as the ultrasonic wave amplitude increases. Surface plasmon resonance is a detection technique whereby the liquid interface is probed by an evanescent wave associated with the reflection of light from an external source and produces an intensity minimum at a narrowly defined resonance angle.

The amplitude and frequency at which the bonds rupture will depend on the type of bond formed and on the substance bonded to the sensor surface. Bond rupture produces a change in surface properties that can be detected and transformed into an electrical signal. The amplitude and/or frequency of waves at which the bond rupture occurs can then be compared to the known amplitude and/or frequency of bond rupture for the test fluid and the presence of the target substance in the test fluid can thereby be determined. In preferred embodiments the waves are ultrasonic waves.

The oscillator may be any suitable device. For example the oscillator could be a mechanical resonator, an acoustic oscillator, a piezoelectric device, or a microcantilever. When the biosensor is immersed in liquid, the oscillator produces waves that vibrate the liquid which in turn vibrate the bio-macromolecules and substances bound to the bio-macromolecules. By vibrating the liquid medium surrounding the sensor surface the problem with damping that occurs when the sensor surface is vibrated is overcome. In preferred embodiments the waves are directed towards the sensor surface.

In a preferred embodiment detection of bond rupture is by surface plasmon resonance. The technique of surface plasmon resonance requires light to be directed at the sensor surface from the side opposite that bound to the bio-macromolecules. The intensity of reflected light is at a minimum at a particular angle for a given wavelength. When the property of the sensor surface changes, for example by bond rupture, so does the angle of minimum reflectance. This change is detected by a detector indicating a change in mass on the sensor surface. In another embodiment detection of bond rupture can be by detecting an acoustic emission caused by bond rupture.

When surface plasmon resonance is used to detect bond rupture the substrate 4 is preferably glass or plastics. These materials are useful because, unlike acoustic emission detection, they require no electrical contacts and are suitable for multiple tests and microfluidic handling of small samples. This can lead to a disposable chip.

In use, as the bio-macromolecule is forms d on the surface and the target substance binds to the bio-macromolecule the surface plasmon resonance detector will show a shift in the resonance angle that corresponds to both specific and non-specific attachment. As the waves are produced and the substances bonded to the bio-macromolecules vibrate the surface plasmon resonance detector will show shifts in the resonance angle, one is expected over a broad range of excitation as the non-specifically bound molecules detach and another, much sharper one, as the bonds between the target substance and the bio-macromolecules rupture.

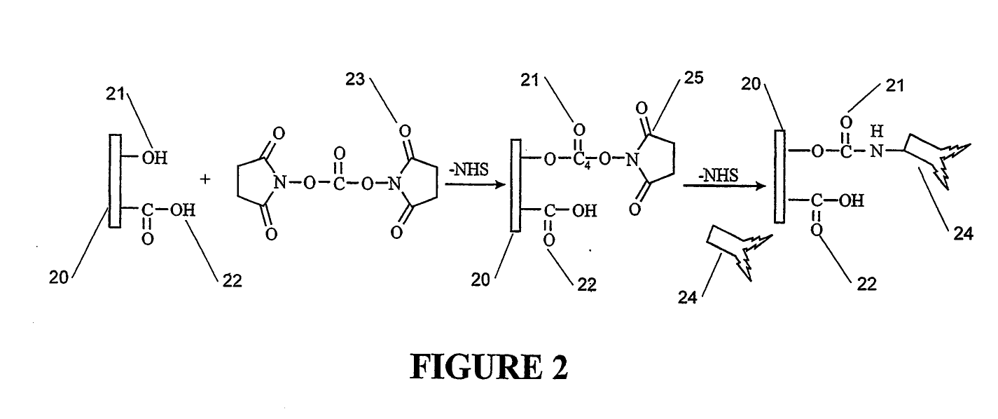

FIG. 2 shows one step for bonding an antibody as bio-macromolecule layer to a sensor surface through the use of a self assembled monolayer. The self assembled monolayer is a mixed monolayer of 11-mercapto-1-undecanol (MUOH) and 16-mercapto-1-hexadecanoic acid (MHA). Both of these chemicals bond to the gold layer of the sensor surface through their sulphur atoms to leave free alcohol and acid groups respectively onto which an antibody can be bonded. A N,N′-disuccinimidyl carbonate (DSC)-activated hydroxyl group is then used as a catalyst to bond a bio-macromolecule to the self assembled monolayer.

In FIG. 2 the sensor surface and bulk of the self assembled monolayer are represented by layer 20. The alcohol group of 11-mercapto-1-undecanol and the acid group of 16-mercapto-1-hexadecanoic acid are shown at 21 and 22 respectively. The N,N′-disuccinimidyl carbonate (DSC) is shown at 23. This group temporarily binds to 21 and/or 22 to form an activated carbonyl centre. When a bio-macromolecule such as an antibody or protein 24 is present it reacts to create a peptide bond thus binding the bio-macromolecule to the self assembled monolayer and thereby to the sensor surface. Other suitable activating agents may be used in place of the N,N′-disuccinimidyl carbonate (DSC) as per standard peptide synthesis techniques.

As shown in FIG. 1 the top layer of the sensor surface is gold to allow binding of a self assembled monolayer to the sensor surface. In an alternative embodiment the top layer of the sensor surface is another metal that allows binding of a self assembled monolayer. For example, the top layer of the sensor surface may be silver. The sensor surface may include further layer(s) that may form part of the detector. For example if a surface plasmon resonance detector is used the sensor surface may be a thin layer of metal deposited on glass or some other transparent material with a refractive index higher than that of the liquid.

There are several techniques for binding a self assembled monolayer to the sensor surface.

Carboxylic acid end groups enable derivatisation of a self assembled monolayer with bio-macromolecules. This process uses light-activated affinity micropatterning using deprotection by UV light through a photomask. This process can be used to produce multi-enzyme scaffolds in which enzyme A passes its product to adjacent enzyme B and so on, cooperating in a localised metabolic chain.

Another technique for forming a self assembled monolayer is dip-pen nanolithography. This technique can be used to construct features as small as 100 to 350 nm. This technique involves coating an atomic force microscopy tip with “ink” such as 11-mercapto-1-undecanol or 16-mercapto-1-hexadecanoic acid. After immersion of dots or lines of 11-mercapto-1-undecanol or 16-mercapto-1-hexadecanoic acid in protein solutions, monolayers or protein adhere to the coated regions of the sensor surface.

The self assembled monolayer and bio-macromolecules may be self assembled lipid membranes. These membranes use phospholipid vesicles which exhibit a natural tendency to fuse and assemble into a continuous single bilayer membrane on silica and several other substrate materials. The advantage of self assembled lipid membranes is that there are only weak interactions between the support and the bio-macromolecule due to van der Waals forces, dipole-dipole interactions or hydrogen bonding. The reversible nature of the binding equilibrium is highlighted by its susceptibility to changes in pH, ionic strength, temperature, etc. The reversible nature of the binding equilibrium allows self repair of potential deficiencies. Self assembled lipid membranes are useful because they mimic biological membranes, even exhibiting lateral fluidity on a wet surface.

The main advantages of forming a self assembled monolayer between the sensor surface and the bio-macromolecule and binding the bio-macromolecule to the self assembled monolayer as shown in FIG. 2 are that the coupling step can be carried out in a neutral buffer and that the resulting uncharged carbamate bond is very stable, so leakage of bound protein is minimised. The proper choice of immobilisation method is important so that the bio-macromolecules retain activity, stability, and specificity on the sensor surface.

Alternatives to the gold surface as a support for the self-assembled monolayer could be silicon or glass. For these surfaces a silane-coupling agents such as HO(CH₂)₁₇SiCl₃ or HO(CH₂)₁₇Si(OCH₃)₃ could be used.

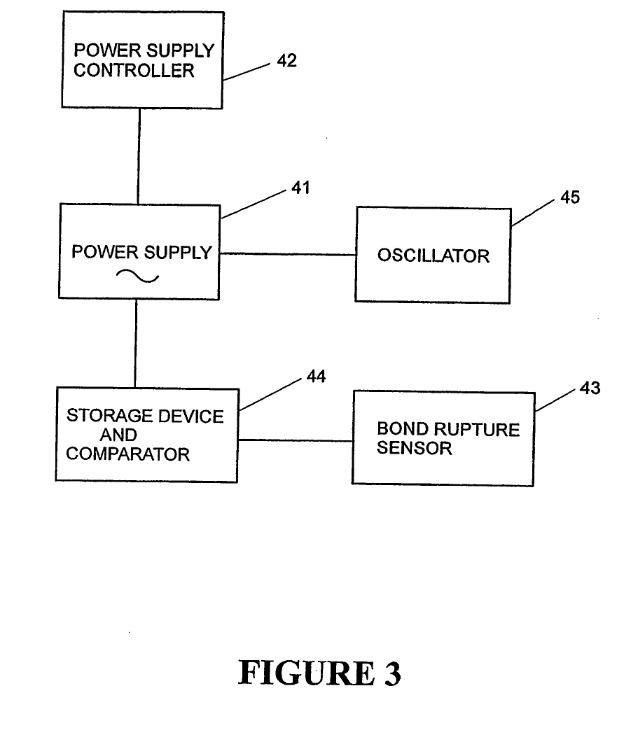

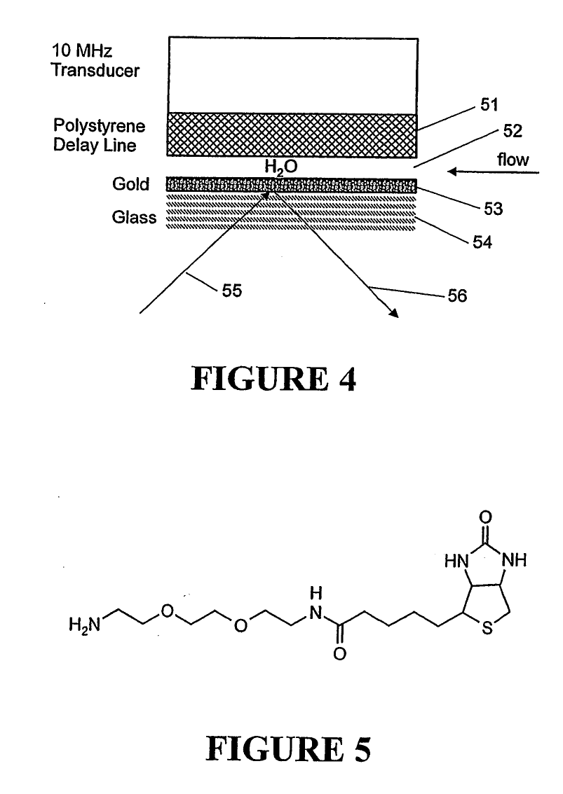

FIG. 3 shows a block diagram of the biosensor of the invention. The biosensor includes sensor surface 40, power supply 41, power supply controller 42, bond rupture sensor 43, oscillator 45 and storage device and comparator 44.

In use, a test fluid is brought into contact with sensor surface 40. The sensor surface includes a layer of bio-macromolecules chosen to bind to a target substance. If the target substance is present in the test fluid it will bind to the bio-macromolecules. Other substances present in the target fluid may also bind to the bio-macromolecules. However, the bio-macromolecules are chosen for their ability to bind to the target substance and as few other substances as possible.

In preferred embodiments at least the sensor surface and oscillator are immersed in liquid. In alternative embodiments the sensor surface and oscillator are not immersed in liquid.

After the sensor surface is brought into contact with the test fluid (shown in FIG. 1 C) waves with varying frequency and/or amplitude are directed at the surface from oscillator 45. Power supply 41 supplies the power to oscillator 45. Power supply 41 may be controlled by power supply controller 42. Alternatively the power supply controller 42 may be built into power supply 41. Power supply controller 42 controls the power supplied by power supply 41 to oscillator 45. Power supply controller 42 may change the rate at which the frequency and/or amplitude of the waves are changed, when voltage is supplied to oscillator 45 and when voltage is to no longer be supplied to oscillator 45.

As the waves are directed towards the surface bond rupture sensor 43 detects when bonds between the bio-macromolecules and substances bound to the bio-macromolecules rupture. As tie frequency and/or amplitude of the waves increases, the force exerted by the waves on substances bound to bio-macromolecules increases and precipitates rupture of the bonds. When bonds between the bio-macromolecules on the bond rupture sensor and substances bonded to the bio-macromolecules rupture, step change in the surface plasmon resonance is produced. In a further embodiment the bond rupture sensor 43 is a piezoelectric substrate such that bond rupture causes an acoustic signal that is transduced into a detectable voltage.

Each substance bonded to the bio-macromolecules of the sensor surface will rupture at different frequencies and/or amplitudes and if more than one different substance is bonded to the bio-macromolecules of the sensor surface there may be more than one rupture event detected by bond rupture sensor 43.

Bond rupture sensor 43 passes an indication of the bond rupture to storage device and comparator 44. The indication may be a voltage level indication or any other suitable indication. The storage device and comparator also receives an indication of the frequency and/or amplitude of waves from oscillator 45 at which the bond rupture occurred from power supply 41. The storage device and comparator then compares the rupture indicator and frequency and/or amplitude of waves at which the rupture occurred to stored data. If the rupture indicator and voltage correspond to data relating to the target substance the storage device and comparator indicates that the target substance is present. Such an indication may be via a monitor or by an audio indication.

### EXAMPLE

FIG. 5 shows an example of one embodiment of biosensor that uses an SPR device to detect bond rupture. The biosensor includes an oscillation source 50, delay line 51, fluid channel 52, surface 53, reflective layer 54, incident light beam 55, and reflected light beam 56. A light detector is also included (not shown) that detects changes on the angle of the reflected light beam 56. In this example oscillator 50 is a 10 MHz transducer that can be connected to a wave form generator.

This example shows both surface immobilisation and bond rupture scanning. These were monitored in situ by integration of both SPR detection and acoustic waveform induction into a thin layer flow cell.

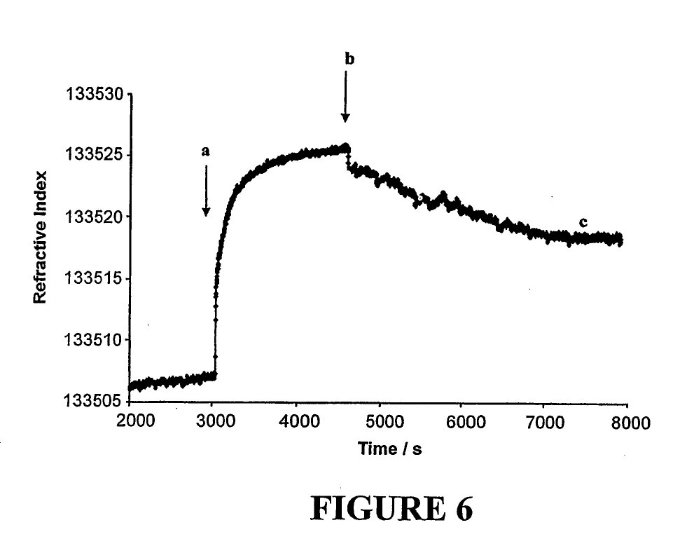

Surface 53, in this example a gold surface, provides a surface on which a self assembled monolayer may be formed. In this example 5 mg of biotin-PEO3-amine was dissolved in 250 mL of 0.1 M phosphate buffered saline (PBS) solution (pH 7.4). The amine and PBS solution was flushed through the bare gold surface 53 at a flow rate of 20 mL/min. FIG. 5 shows the molecular structure of the biotin-PEO3-amine. Immobilisation of the biotin-PEO3-amine onto the gold surface 53 was monitored by the SPR detector as shown in FIG. 6. As can be seen in FIG. 6, the refractive index detected by the SPR detector shows very little change until a time just after 3000 seconds and marked as “a” on this figure where the biotin-PEO3-amine solution was applied. During the application time, which occurs until a point marked as “b” in FIG. 6, the refractive index detected by the SPR detector changes. The initial change of the refractive index detected by the SPR detector is rapid and this tails off towards point “b” suggesting that as much biotin-PEO3-amine as possible has been bonded to the gold.

At point b shown on FIG. 6 a 0.1M PBS solution was introduced to rinse off loosely bound biotin-PEO3-amine. This is shown in FIG. 6 where after point b the refractive index changes as loose amine is rinsed from the biotinylated self-assembled monolayer (SAM). The primary-amine groups bind very strongly to the gold surface to produce a biotinylated SAM. The area marked “c” on FIG. 6 indicates a new base line refractive index after the buffer solution has been passed through the biosensor.

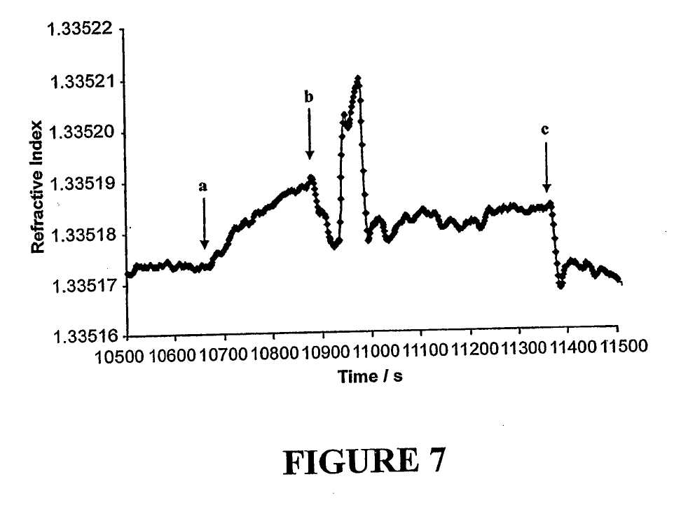

A solution of 10 μg of streptavidin dissolved in 100 mL of 0.1M PBS solution (pH 7.4) was flushed through the biosensor at a rate of 20 mL/min. The streptavidin solution bonded to the biotinylated SAM during the flushing process. The flushing process begins at the position marked “a” on FIG. 7. Interaction between the Biotin and streptavidin was monitored by the SPR detector as shown in FIG. 7.

FIG. 7 shows the binding of the streptavidin to the biotin between the positions marked “a” and “b”. After point b the biosensor was flushed with a solution of 0.1 M PBS. There are some fluctuations caused by releasing non-specific adsorption of streptavidin on the biotinylated SAM, due to switching back to the normal PBS running buffer. This is shown between points b and c on FIG. 7. By point c a new baseline refractive index is established the sensor now contains a self assembled monolayer to which a target species and in has been bound.

Oscillations are then produced by an oscillation source, in this example a 10 MHz transducer shown at 50 on FIG. 5, with a waveform at 1 Vpp to generate ultrasonic energy. As can be seen at point c on FIG. 7 there is a step change of the refractive index due to the dissociation of the streptavidin from the biotinylated SAM.

The example shows that the biosensor of the invention can rupture bonds between a target substance and a self-assembled monolayer. The example further shows that the bond rupture can be detected.

Although the biosensor described includes only one sensor surface, biosensors may contain a number of surface areas each with bio-macromolecules provided to bind with different target substances. The surface areas may be provided on a single base surface. In this way one biosensor can be provided that tests for a range of target substances instead of requiring different biosensors for each substance.

The foregoing describes the invention including preferred forms thereof. Alterations and modifications as will be obvious to those skilled in the art are intended to be incorporated in the scope hereof.

1. A biosensor comprising;
a surface onto which bio-macromolecules are bound the surface and bio-macromolecules being immersed in liquid,
a bond rupture detector associated with the surface and arranged to detect the rupture of bonds between the bio-macromolecules and a target substance, and
an oscillator associated with the liquid and spaced from the surface and arranged to produce oscillations in the liquid to cause bonds between the bio-macromolecules and the target substance to rupture.

2. A biosensor as claimed in claim 1 wherein the bond rupture detector is a surface plasmon resonance detector that detects, when bonds between the bio-macromolecules and the target substance rupture, a change in the angle of reflected light that has minimum reflectance.

3. A biosensor as claimed in claim 1 wherein the bond rupture detector detects acoustic emissions produced when bonds between the bio-macromolecules and target substance rupture.

4. A biosensor as claimed in any one of claims 1 to 3 wherein the oscillator produces oscillations that are ultrasonic in frequency.

5. A biosensor as claimed in any one of claims 1 to 4 wherein the oscillator is arranged to provide oscillations at a predetermined frequency.

6. A biosensor as claimed in any one of claims 1 to 4 wherein the oscillator is arranged to provide oscillations over a range of frequencies.

7. A biosensor as claimed in any one of claims 1 to 6 wherein the amplitude of the oscillations produced by the oscillator is constant,

8. A biosensor as claimed in any one of claims 1 to 6 wherein the amplitude of tile oscillations produced by the oscillator changes.

9. A biosensor as claimed in any one of claims 1 to 8 further comprising a self assembled monolayer adapted to bind the bio-macromolecules to the surface.

10. A biosensor as claimed in any one of claims 1 to 9 wherein the surface is coated in gold or silver.

11. A biosensor as claimed in any one of claims 1 to 10 comprising more than one surface, each surface provided with bio-macromolecules that bind to different target substances.

12. A method of detecting a target substance comprising the steps of;
providing a biosensor including at least one surface onto which bio-macromolecules are bound, a bond rupture detector associated with the surface and arranged to detect the rupture of bonds between the bio-macromolecules and a target substance, and an oscillator separate from the surface and arranged to produce waves to cause bonds between the bio-macromolecules and the target substance to rupture,
bringing the biosensor into contact with a test fluid that may contain the target substance,
generating oscillations with the oscillator directed at the surface and
detection whether any bonds rupture with the detector, and
comparing a parameter of the oscillator when the bonds rupture with stored data and wherein
during operation of the biosensor the surface of the biosensor and the oscillator are immersed in liquid.
